(19) United States
(12) Patent Application Publication
(10) Pub. No.: US 2013/0244284 A1
(43) Pub. Date: Sep. 19, 2013

(54) METHOD FOR SUBJECTING SOLID BIOMASS TO SACCHARIFICATION PRETREATMENT, APPARATUS THEREFOR, AND METHOD FOR SACCHARIFICATION OF SOLID BIOMASS

(71) Applicants: Masashi Machida, Ichihara-shi (JP); Tetsuya Fukunaga, Sodegaure-shi (JP); Kozo Morimitsu, Chiyoda-ku (JP); Shigeru Sakashita, Meguro-ku (JP); Yasuo Torikata, Nikko-shi (JP); Akihiko Kosugi, Tsukuba-shi (JP); Yutaka Mori, Tsukuba-shi (JP); Yoshinori Murata, Tsukuba-shi (JP); Takamitsu Arai, Tsukuba-shi (JP)

(72) Inventors: Masashi Machida, Ichihara-shi (JP); Tetsuya Fukunaga, Sodegaure-shi (JP); Kozo Morimitsu, Chiyoda-ku (JP); Shigeru Sakashita, Meguro-ku (JP); Yasuo Torikata, Nikko-shi (JP); Akihiko Kosugi, Tsukuba-shi (JP); Yutaka Mori, Tsukuba-shi (JP); Yoshinori Murata, Tsukuba-shi (JP); Takamitsu Arai, Tsukuba-shi (JP)

(73) Assignees: Japan International Research Center for Agricultural Sciences; Idemitsu Kosan Co., Ltd.

(21) Appl. No.: 13/878,960

(22) PCT Filed: Oct. 12, 2011
(86) PCT No.: PCT/JP2011/073396

§ 371 Date: Jun. 4, 2013

(30) Foreign Application Priority Data
Oct. 12, 2010 (JP) 2010-229512

(51) Int. Cl. C12P 19/00

(52) U.S. Cl. CPC C12P 19/00
USPC 435/72; 435/300.1

(57) ABSTRACT

Solid biomass having a moisture content in a range of 30 to 95 mass % is brought into contact with ammonia gas at a pressure in a range of 0.5 to 4 MPa and held at 50 to 200 degrees C. Subsequently, the solid biomass is rapidly decompressed to be blasted. The ammonia gas evaporated by decompression is recovered and recycled. The ammonia gas can be brought into contact with the entire solid biomass evenly and in a short time. Since absorption heat of the ammonia gas is generated, energy consumption required for additional heating can be reduced.

### TECHNICAL FIELD

The present invention is related to a method for subjecting solid biomass to a saccharification pretreatment, an apparatus therefor, and a method for saccharification of the solid biomass subjected to the saccharification pretreatment.

### BACKGROUND ART

In recent years, an effective use of solid biomass, for instance, manufacturing of bioethanol fuel from solid biomass, has been developed.

Manufacturing of bioethanol fuel from solid biomass requires hydrolysis and saccharification of the solid biomass. As a pretreatment for promoting saccharification of the solid biomass, in order to make a diastatic enzyme readily accessible to cellulose, treatments such as separation of hemicellulose or lignin and non-crystallization of crystalline cellulose are performed. For efficient saccharification, only hemicellulose capable of relatively easily reacting is occasionally converted into sugar or oligosaccharide under moderate conditions.

As the method for the pretreatment of the solid biomass, specifically, a vapor blasting method, a method using concentrated sulfuric acid, a method using dilute sulfuric acid, a method using hot-compressed water, an ammonia blasting method and the like (see Patent Literatures 1-10) are widely used.

However, since acid is used in the method using dilute sulfuric acid and the method using concentrated sulfuric acid, particularly, excessive decomposition of hemicellulose easily occurs. Moreover, in the vapor blasting method and the method using hot-compressed water, since the solid biomass is treated under high temperatures and high pressures, excessively decomposed product is easily produced.

Thus, in the method using dilute sulfuric acid, the method using concentrated sulfuric acid, the vapor blasting method or the method using hot-compressed water, since a yield of sugar is low because of excessive decomposition, improvement in a yield of bioethanol is desired.

On the other hand, as an ammonia blasting method without excessive decomposition, a method using liquid ammonia or an ammonia aqueous solution (Patent Literatures 1-7, 9 and 10), a method using ammonia gas (Patent Literatures 1 and 8) and the like are known.

Patent Literature 1 discloses a method for enhancing cellulose digestibility and protein availability of alfalfa to enhance alfalfa as feed for animals. In this method, alfalfa is kept in contact with ammonia for less than about one hour at a treatment pressure of about 0.97 MPa (140 psia) to about 1.24 MPa (180 psia) at a treatment temperature of about 25 degrees C.

Patent Literature 2 discloses a method for increasing reactivity of a cellulose material. In this method, liquid ammonia and a cellulose-containing material are brought into contact with each other in a pressure vessel. The cellulose-containing material and the liquid ammonia are kept mixed in the pressure vessel at a temperature higher than atmospheric temperature surrounding the pressure vessel and at least under vapor pressure of the liquid ammonia, until the elapse of a sufficient time for the cellulose-containing material to be substantially totally immersed in the liquid ammonia. Subsequently, the liquid ammonia is boiled and rapidly decompressed in the pressure vessel to a state sufficient for blasting or tearing a cellulose fiber structure.

Patent Literature 3 discloses a method for treating lignocellulose-based biomass with ammonia water. In this method, the lignocellulose-based biomass is mixed with ammonia water supplied by an ammonia water supplier, and then the mixture is heated for boiling, in which the boiled ammonia water is swollen to swell the biomass. By an alkaline treatment using the ammonia water, lignin that inhibits an enzyme saccharification reaction is removed. Ammonia gas evaporated by heating is dissolved in water and recovered as ammonia water.

Patent Literature 4 discloses a method for treating a lignocellulose material with the liquid ammonia. In this method, a lignocellulose material having a moisture content of 10% to 80% is brought into contact with the liquid ammonia in a barrel set at about 30 degrees C. to 100 degrees C. Next, pressure is rapidly decreased, whereby the liquid ammonia is swollen into gas from a liquid to swell the lignocellulose material.

Patent Literatures 5 and 6 disclose a method for treating cellulose with liquid ammonia. In the method, cellulose having a moisture content of less than 12 mass % is brought into contact with liquid ammonia at a temperature of about 25 degrees C. to 85 degrees C. under a pressure of 0.5 MPa (5 bar) to 4.6 MPa (46 bar) exceeding atmospheric pressure. A pressure of the mixture of cellulose and liquid ammonia is released and lowered at least to a 0.5 MPa (5 bar) for less than one second, thereby explosively rapidly increasing an effective volume of polysaccharide-liquid ammonia system.

Patent Literature 7 discloses a method for treating cellulose with liquid ammonia. In this method, cellulose is compressed in a pressure vessel to atmospheric pressure or more and is brought into contact with liquid ammonia, and then the pressure vessel is rapidly decompressed to atmospheric pressure for continuous swelling. In this method, cellulose pulp containing alpha cellulose of 92 mass % or more is brought into contact with ammonia at the room temperature or more, thereby swelling ammonia. After the swelling of ammonia, while the minimum content of ammonia enough to maintain an active state derived from the reaction of ammonia is left, the rest of ammonia remaining in the pressure vessel is removed. The residual ammonia required for maintaining the active state is replaced with a swelling agent or a containing agent.

Patent Literature 8 discloses a method for bringing a cellulose-containing substance into contact with liquid ammonia or gaseous ammonia at atmospheric temperature under a pressure higher than atmospheric pressure. In this method, after the cellulose-containing substance is brought into contact with ammonia, pressure is rapidly decreased to boil ammonia, thereby blasting a transition structure of the cellulose-containing substance. Ammonia is recovered for recycling.

Patent Literature 9 discloses a method for bringing cellulose into contact with liquid ammonia under compression in a closed room and then rapidly decreasing a pressure applied on ammonia.

Patent Literature 10 discloses a method for bringing cellulose into contact with liquid ammonia, which has a sufficient amount to wet a surface of cellulose, at 25 degrees C. or more under a pressure higher than atmospheric pressure. After the contact, the pressure is explosively rapidly decreased to 0.5 MPa (5 bar).

### CITATION LIST

### Patent Literature(s)

Patent Literature 1: Specification of U.S. Pat. No. 4,600,590
Patent Literature 2: Specification of U.S. Pat. No. 5,037,663
Patent Literature 3: JP-A-2010-115162
Patent Literature 4: JP-T-2002-513041
Patent Literature 5: JP-T-10-505130
Patent Literature 6: JP-A-2002-161101
Patent Literature 7: JP-A-7-102001
Patent Literature 8: JP-A-58-79001
Patent Literature 9: JP-A-2000-513042
Patent Literature 10: JP-A-11-504071

### SUMMARY OF THE INVENTION

### Problems to be Solved by the Invention

However, in the method using liquid ammonia or the ammonia aqueous solution as described in Patent Literatures 2 to 7, 9 and 10, infiltration of the liquid ammonia or the ammonia aqueous solution into the solid biomass is time-consuming, so that a treatment efficiency is difficult to improve. Moreover, since a large energy is required in compressing and heating the liquid ammonia or the ammonia aqueous solution, decrease in energy for the treatment has been desired. Further, since the entire solid biomass is infiltrated in the liquid ammonia or the ammonia aqueous solution, a great amount of the liquid ammonia or the ammonia aqueous solution is required.

On the other hand, the method using ammonia gas as described in Patent Literatures 1 and 8 is conducted at the room temperature and at a relatively low pressure of about 0.34 MPa (50 psig). Accordingly, ammonia gas is not sufficiently infiltrated, so that improvement in a sugar yield is not expected.

In view of the above, the invention is related to: a saccharification pretreatment method necessary for reducing a used amount of ammonia while suppressing excessive decomposition and efficiently saccharifying solid biomass; an apparatus for the method; and a method for saccharification of the solid biomass.

### Means for Solving the Problems

According to an aspect of the invention, a saccharification pretreatment method of solid biomass includes: contacting moisture-containing solid biomass with ammonia gas at a pressure higher than atmospheric pressure to dissolve the ammonia gas into moisture of the solid biomass; decompressing the solid biomass contacted with the ammonia gas to a pressure lower than a pressure in the contacting with the ammonia gas to evaporate the ammonia gas dissolved in the moisture of the solid biomass; and separating to recover the ammonia gas evaporated in the decompressing from the solid biomass.

In the above aspect of the invention, preferably, in the contacting of the solid biomass with the ammonia gas, the solid biomass is fed into a pressure vessel, the ammonia gas is compressed and supplied into the pressure vessel in which the solid biomass is fed, and the solid biomass and the ammonia gas are contacted with each other at a pressure higher than atmospheric pressure.

In the above aspect of the invention, the solid biomass preferably has a moisture content in a range of 30 mass % to 95 mass %.

In the aspect of the invention, the ammonia gas preferably has a moisture content of 10 mass % or less.

In the aspect of the invention, in the contacting of the solid biomass with the ammonia gas, the solid biomass is preferably contacted with the ammonia gas at a pressure in a range of 0.5 MPa to 4 MPa.

In the aspect of the invention, in the contacting of the solid biomass with the ammonia gas, before performing the decompressing, a temperature of the solid biomass is preferably set in a range of 50 degrees C. to 200 degrees C.

In the aspect of the invention, in the decompressing, the solid biomass is preferably rapidly decompressed to be blasted.

In the aspect of the invention, in the decompressing, the pressure of the solid biomass is preferably decompressed to a pressure lower than atmospheric pressure.

According to the aspect of the invention, the saccharification pretreatment method preferably further includes recovering the ammonia gas by heating to dry the solid biomass from which the ammonia gas is separated in the separating and separating ammonia adsorbed to the solid biomass.

In the aspect of the invention, the solid biomass is preferably a cellulose-containing biomass.

In the aspect of the invention, the solid biomass is preferably a starch-containing biomass.

According to another aspect of the invention, a saccharification pretreatment apparatus of solid biomass includes: an ammonia gas contact section that contacts a moisture-containing solid biomass with ammonia gas at a pressure higher than atmospheric pressure to dissolve the ammonia gas into moisture of the solid biomass; a decompressor that decompresses the solid biomass contacted with the ammonia gas in the ammonia gas contact section to a pressure lower than a pressure in the ammonia gas contact section to evaporate the ammonia gas dissolved in the moisture of the solid biomass; and an ammonia gas separating section that separates to recover the ammonia gas evaporated in the decompressor from the solid biomass.

According to still another aspect of the invention, a saccharification method of the solid biomass according to the above aspect of the invention is treated with the saccharification pretreatment method of solid biomass according to the above aspect of the invention.

In the above aspect of the invention, the solid biomass is brought into contact with ammonia gas under a pressure higher than atmospheric pressure to dissolve the ammonia gas into moisture of the solid biomass. When the ammonia gas is dissolved, since the ammonia gas is drawn into the moisture of the solid biomass, the ammonia gas can be brought into contact with an entirety of the solid biomass evenly and in a short time. Accordingly, a necessary amount of the ammonia gas can be easily minimized. Further, since absorption heat of the ammonia gas is generated to heat the solid biomass, energy consumption for additionally heating the solid biomass to be modified so as to be easily saccharified in a posterior process can be suppressed. A reaction temperature for heating the solid biomass by absorption heat can be easily determined by controlling an amount of the moisture of the solid biomass and the pressure when bringing the solid biomass into contact with the ammonia gas. Accordingly, the saccharification pretreatment of the solid biomass can be controlled simply, easily and stably.

By decompressing after contact of the solid biomass and the ammonia gas and recovering the ammonia gas dissolved in the moisture of the solid biomass through vaporization, recycling of the ammonia gas becomes possible and costs for the saccharification pretreatment of the solid biomass is reducible.

### BRIEF DESCRIPTION OF DRAWINGS

FIG. 1 is a block diagram showing a schematic structure of a saccharification pretreatment apparatus of solid biomass according to an exemplary embodiment of the invention.

### DESCRIPTION OF EMBODIMENT(S)

A saccharification pretreatment apparatus of solid biomass according to an exemplary embodiment of the invention will be described below with reference to the attached drawings.

### Structure of Apparatus

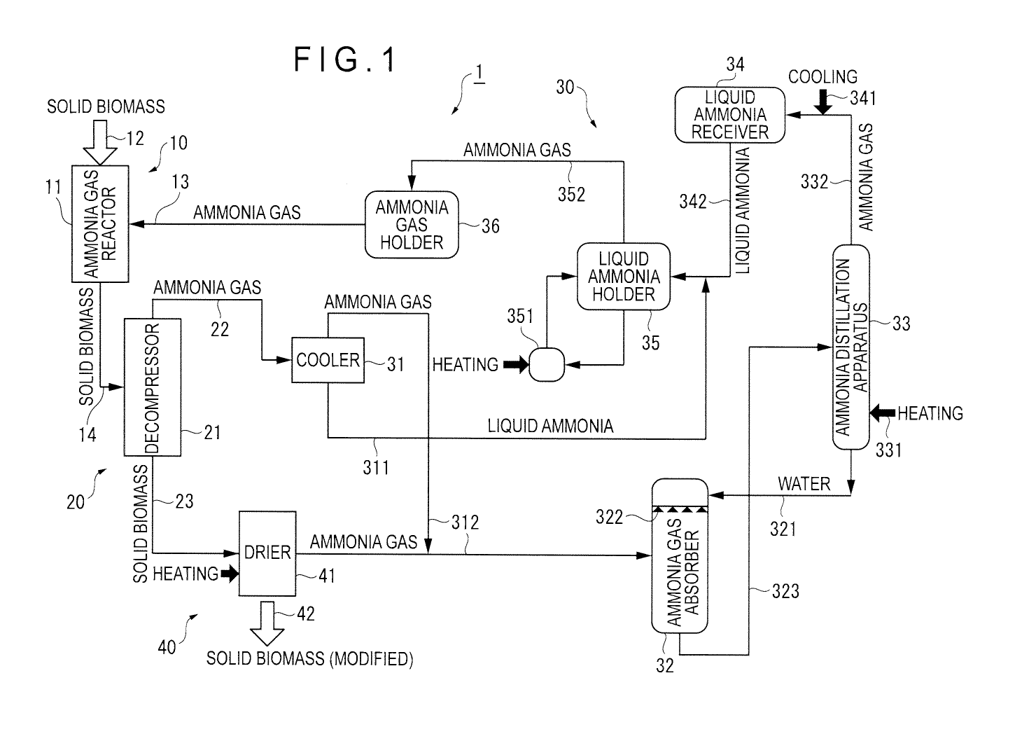

In FIG. 1, a saccharification pretreatment apparatus 1 performs a pretreatment for modifying solid biomass for a saccharification treatment.

The saccharification pretreatment apparatus 1 includes: an ammonia gas contact section 10; a decompression section 20; an ammonia gas recovery section 30 (an ammonia gas separating section); an ammonia gas removing section 40; a controller (not shown); and the like.

Herein, as the solid biomass, for instance, a cellulose-based biomass, a starch-based biomass, and a mixed biomass thereof are usable. Examples of the cellulose-based biomass include a trunk and an empty fruits bunch of a palm tree, bagasse, rice straw, wheat straw, corn residue (e.g., corn stover, corn cob, corn hull), pericarp and shell of Jatropha, and wood chip. Examples of the starch-based biomass include corn and rice. Examples of the mixed biomass include cassava pulp.

A moisture content of the solid biomass is preferably in a range of 30 mass % to 95 mass %. Herein, the moisture content refers to mass percentage of a value obtained by dividing a mass of water by a mass of the solid biomass containing moisture. When the moisture content of the solid biomass is less than 30 mass %, an absolute amount of the ammonia gas to be dissolved is decreased, which may cause an insufficient modification of the solid biomass for efficiently saccharifying the solid biomass in a posterior process. On the other hand, when the moisture content of the solid biomass exceeds 95 mass %, free water is contained in the solid biomass. Into the free water, the ammonia gas is dissolved to reduce the absolute amount of the ammonia gas contributing to the modification of the solid biomass, so that the modification of the solid biomass may become insufficient.

The ammonia gas contact section 10 brings moisture-containing solid biomass into contact with the ammonia gas under a pressure higher than atmospheric pressure to dissolve the ammonia gas into the moisture of the solid biomass.

The ammonia gas contact section 10 includes an ammonia gas reactor 11, a material feeding section 12, an ammonia gas supply pipe 13, and a reaction biomass discharge channel 14.

The ammonia gas reactor 11 is a pressure-resistant and heat-resistant vessel having an inner pressure adjustable to a pressure higher than atmospheric pressure and having heat-retention performance. The ammonia gas reactor 11 includes a pressure sensor (not shown) that measures the inner pressure, a temperature sensor (not shown) that measures a temperature of an inside of the ammonia gas reactor 11, and a heating unit (not shown) (e.g., a heater) that heats the inside as needed.

The material feeding section 12 is provided to the ammonia gas reactor 11 and feeds the moisture-containing solid biomass into the ammonia gas reactor 11.

One end of the ammonia gas supply pipe 13 is connected to the ammonia gas reactor 11. The ammonia gas supply pipe 13 supplies the ammonia gas into the ammonia gas reactor 11.

Herein, the supplied ammonia gas preferably has a moisture content of 10 mass % or less, particularly preferably 5 mass % or less. When the moisture content exceeds 10 mass %, an amount of heat generation of absorption heat generated when the ammonia gas and the solid biomass are in contact with each other is decreased, which may cause increase in energy consumption because of heating by a heating unit. Further, when the moisture content exceeds 10 mass %, sufficient modification of the solid biomass by ammonia may not be obtainable. Ammonia gas having a purity as high as a certain level (i.e., having a moisture content lower than a certain level) requires increased costs while exhibiting not so largely different modification effects. Accordingly, such ammonia gas in a wide distribution, at a low price, and with a moisture content of 5 mass % or less may be sufficiently usable. As an ammonia gas source, ammonia water and liquid ammonia in addition to the ammonia gas are usable as needed.

One end of the reaction biomass discharge channel 14 is connected to the ammonia gas reactor 11. The reaction biomass discharge channel 14 discharges the solid biomass contacted with the ammonia gas out of the ammonia gas reactor 11.

The decompression section 20 decompresses the solid biomass in contact with the ammonia gas in the ammonia gas contact section 10 to a pressure lower than a pressure in the ammonia gas contact section 10, thereby evaporating the ammonia gas dissolved in the moisture of the solid biomass. The decompression section 20 includes a decompressor 21.

The decompressor 21 is connected to the reaction biomass discharge channel 14 of the ammonia gas reactor 10 and receives the solid biomass in contact with the ammonia gas from the ammonia gas reactor 11. The decompressor 21 has a pressure-resistant structure in which an inner pressure is adjustable to a pressure lower than atmospheric pressure. The decompressor 21 includes a pressure sensor (not shown) that measures the inner pressure.

The decompressor 21 is connected with an ammonia gas discharge channel 22 that discharges the evaporated ammonia gas out of the decompressor 21, and a discharge channel 23 that discharges the solid biomass, from which the ammonia gas was evaporated, out of the decompressor 21.

The ammonia gas recovery section 30 separates and recovers the ammonia gas evaporated in the decompressor 21 from the solid biomass. The ammonia gas recovery section 30 includes a cooler 31, an ammonia gas absorber 32, an ammonia distillation apparatus 33, a liquid ammonia receiver 34, a liquid ammonia holder 35, and an ammonia gas holder 36.

The cooler 31 is connected to the ammonia gas discharge channel 22 and is supplied with the ammonia gas evaporated in the decompressor 21. The cooler 31 cools the ammonia gas transferred from the decompressor 21 and liquefies the ammonia gas into liquid ammonia. The cooler 31 includes a liquid ammonia discharge channel 311 that discharges liquid ammonia, and an ammonia gas discharge channel 312 that discharges the remaining non-liquefied ammonia gas.

The ammonia gas absorber 32 is connected to the ammonia gas discharge channel 312 and makes water absorb the ammonia gas transferred from the cooler 31. The ammonia gas absorber 32 includes: a water supply channel 321; a water sprinkler 322 that sprinkles water supplied from the water supply channel 321; and an ammonia aqueous solution discharge channel 323 that discharges an ammonia aqueous solution containing the dissolved ammonia gas out of the ammonia gas absorber 32.

The ammonia distillation apparatus 33 is connected to the ammonia aqueous solution discharge channel 323 and distills the ammonia aqueous solution transferred from the ammonia gas absorber 32 to separate the ammonia aqueous solution into water and ammonia gas. The ammonia distillation apparatus 33 includes a heater 331 for distilling the ammonia aqueous solution. The ammonia distillation apparatus 33 is connected with a water supply channel 321 that returns the separated water to the ammonia gas absorber 32. Further, the ammonia distillation apparatus 33 includes a gas discharge channel 332 that discharge the separated ammonia gas.

The liquid ammonia receiver 34 is connected to the gas discharge channel 332 and supplied with the ammonia gas separated in the ammonia distillation apparatus 33 to liquefy the ammonia gas into liquid ammonia. The liquid ammonia receiver 34 includes a cooler 341 that cools to liquefy the supplied ammonia gas. The liquid ammonia receiver 34 includes a liquid discharge channel 342 that discharges the liquid ammonia.

The liquid ammonia holder 35 is connected to the liquid ammonia discharge channel 311 and the liquid discharge channel 342 and stores the liquid ammonia transferred from the cooler 31 and the liquid ammonia receiver 34. Further, the liquid ammonia holder 35 evaporates the stored liquid ammonia into ammonia gas as needed. The liquid ammonia holder 35 includes a heating section 351 that vaporizes the liquid ammonia by heat. A pressure of the ammonia gas to be evaporated is set depending on heating conditions of the heating section 351. The liquid ammonia holder 35 includes a gas flow channel 352 through which the evaporated ammonia gas flows down to be supplied to the ammonia gas contact section 10.

The ammonia gas holder 36 is connected to the gas flow channel and stores the ammonia gas supplied from the liquid ammonia holder at a pressure higher than atmospheric pressure. The ammonia gas holder is connected with an ammonia gas supply pipe and supplies the stored ammonia gas to the ammonia gas reactor.

In the ammonia gas removing section 40, the ammonia gas decompressed and evaporated in the decompression section 20 heats to dry the solid biomass separated by the ammonia gas recovery section 30, thereby separating and recovering ammonia adsorbed to the solid biomass. The ammonia gas removing section 40 includes a drier 41.

The drier 41 is connected to the discharge channel 23 of the decompression section 20 and is supplied from the decompressor 21 with the solid biomass from which the ammonia gas is separated. The drier 41 heats the supplied solid biomass to remove the ammonia gas adsorbed to the solid biomass as well as the moisture. The drier 41 is connected to the ammonia gas discharge channel 312 that supplies the ammonia gas removed from the solid biomass to the ammonia gas absorber 32. The drier 41 further includes a supply channel 42 that supplies the solid biomass modified by contact with the dried ammonia to a saccharification device (not shown) for the saccharification treatment in a posterior process.

As the saccharification treatment in the posterior process, various saccharification methods such as enzyme saccharification, saccharification by hot-compressed water, saccharification by sulfuric acid catalysts, and saccharification by solid catalysts are usable.

The controller controls whole operations of the saccharification pretreatment apparatus 1.

Specifically, the controller controls: flows of the solid biomass, ammonia gas, liquid ammonia, ammonia aqueous solution and water; flow rates thereof, given pressures and temperatures; and the saccharification pretreatment of the solid biomass.

### Saccharification Pretreatment Method

Next, a saccharification pretreatment method for modifying the solid biomass by the saccharification pretreatment apparatus 1 will be described.

Firstly, the solid biomass is fed from the material feeding section into the ammonia gas reactor. At this time, a pressure in the ammonia gas reactor may be kept at atmospheric pressure.

The controller supplies the ammonia gas stored in the ammonia gas holder 36 at a pressure higher than atmospheric pressure to the ammonia gas reactor 11 through the ammonia gas supply pipe 13. Herein, for supplying the ammonia gas, the controller heats as needed to vaporize the liquid ammonia stored in the liquid ammonia holder 35, and sequentially stores the ammonia gas in the ammonia gas holder 36 at the following pressure higher than atmospheric pressure.

The pressure for supplying the ammonia gas is determined under the condition that the solid biomass is in contact with the ammonia gas at a pressure higher than atmospheric pressure.

Specifically, the solid biomass is preferably brought into contact with the ammonia gas at a pressure in a range of 0.5 MPa to 4 MPa, particularly preferably, of 0.7 MPa to 2 MPa. Herein, since heating to high temperatures is required to provide a pressure higher than 4 MPa, energy loss is increased. Moreover, when the solid biomass is heated to high temperatures, hemicellulose may be excessively decomposed and a fermentation inhibitor may be formed, thereby decreasing a production efficiency of ethanol by fermentation in a posterior process of saccharification. Thus, when the pressure is higher than 4 MPa, an efficient saccharification pretreatment may not be performed. On the other hand, when the pressure is lower than 0.5 MPa, since a sufficient amount of the ammonia gas relative to the solid biomass is not supplied, effects of the saccharification pretreatment may not be fully exhibited.

Moreover, the ammonia gas is dissolved in the moisture of the solid biomass by the contact between the solid biomass and the ammonia gas. When the ammonia gas is dissolved, absorption heat is generated to heat the solid biomass. The controller heats as needed the solid biomass by the heating unit in case of shortage of absorption heat such that the temperature of the solid biomass falls within a range of 50 degrees C. to 200 degrees C., particularly of 50 degrees C. to 170 degrees C.

Herein, when the temperature of the solid biomass becomes lower than 50 degrees C., modification of the solid biomass by the ammonia gas may become insufficient. On the other hand, when the temperature of the solid biomass becomes higher than 200 degrees C., the solid biomass may be excessively decomposed to form an excessively decomposed product, thereby reducing a fermentation efficiency in the posterior process.

Particularly, when the solid biomass is starch-based biomass, handling of the solid biomass may become difficult since the solid biomass is dissolved at high temperatures. Accordingly, it is particularly preferable to adjust the temperature of the solid biomass in a range of 50 degrees C. to 70 degrees C. When the solid biomass is cellulose-based biomass, it is particularly preferable to adjust the temperature of the solid biomass in a range of 70 degrees C. to 130 degrees C.

The solid biomass and the ammonia gas are in contact with each other at the pressure higher than atmospheric pressure and in a predetermined range of the temperature. This contact state is maintained for a predetermined time (e.g., duration of one minute to three hours, particularly of three minutes to two hours).

In other words, since the ammonia gas is dissolved in the moisture of the solid biomass, even in a relatively short duration of time as long as one minute, the ammonia gas can be brought into even contact with the entire solid biomass. When the duration of time in contact is shorter than one minute, modification of the solid biomass may become insufficient. On the other hand, even in contact for the duration of time longer than three hours, the modification of the solid biomass is not so different and a treatment efficiency may not be improved.

Subsequently, the controller supplies the solid biomass in contact with the ammonia gas to the decompressor 21 through the reaction biomass discharge channel 14. Although the inner pressure of the decompressor 21 may be equal to atmospheric pressure, the inner pressure thereof is preferably adjusted to a pressure lower than atmospheric pressure.

The solid biomass supplied from the ammonia gas reactor 11 under a pressure higher than atmospheric pressure to the decompressor 21 becomes rapidly decompressed. With this treatment, the ammonia gas dissolved in the moisture of the solid biomass is rapidly evaporated to blast the solid biomass.

The ammonia gas evaporated by decompression is recovered by the ammonia gas recovery section 30. In other words, the ammonia gas evaporated in the decompressor 21 is supplied to the cooler 31 through the ammonia gas discharge channel 22. The cooler 31 cools to liquefy the ammonia gas to liquid ammonia, supplies the liquid ammonia to the liquid ammonia holder 35 through the liquid ammonia discharge channel 311, and stores the liquid ammonia in the liquid ammonia holder 35.

The ammonia gas left non-liquefied in the cooler 31 is supplied to the ammonia gas absorber 32 through the ammonia gas discharge channel 312 and is absorbed in water. The ammonia aqueous solution produced in the ammonia gas absorber 32 is distilled in the ammonia distillation apparatus 33 through the ammonia aqueous solution discharge channel 323. The separated ammonia gas is supplied to the liquid ammonia receiver 34 through the gas discharge channel 332 and is liquefied into liquid ammonia. The obtained liquid ammonia is concentrated so as to have a moisture content of 5 mass % or less. The liquid ammonia is supplied to the liquid ammonia holder 35 through the liquid discharge channel 342 and stored in the liquid ammonia holder 35.

On the other hand, the solid biomass from which the ammonia gas is evaporated in the decompressor 21 is supplied to the ammonia gas removing section 40.

In other words, the solid biomass is supplied from the decompressor 21 to the drier 41 through the discharge channel 23 and dried by heat. By this drying by heat, the ammonia gas adsorbed to the solid biomass is removed together with the moisture. For an efficient removal of the ammonia gas, hot vapor is usable in this drying by heat. The solid biomass dried by heat is supplied as modified solid biomass to a saccharification device (not shown) in the posterior process through the supply channel 42 and is subjected to a saccharification treatment.

The ammonia gas removed together with the moisture in the drier 41 is supplied to the ammonia gas absorber 32 through the ammonia gas discharge channel 312 and is processed with the ammonia gas from the cooler 31.

### WORKING EFFECTS OF EMBODIMENT(S)

As described above, the solid biomass is brought into contact with the ammonia gas at the pressure higher than atmospheric pressure to dissolve the ammonia gas into moisture of the solid biomass. When the ammonia gas is dissolved, since the ammonia gas becomes drawn into the moisture of the solid biomass, the ammonia gas can be brought into contact with the entire solid biomass evenly and in a short time. Accordingly, a necessary amount of the ammonia gas can be easily minimized. Further, since absorption heat of the ammonia gas is generated to heat the solid biomass, energy consumption for additionally heating the solid biomass to be modified so as to be easily saccharified in the post-process can be suppressed. A reaction temperature for heating the solid biomass by the absorption heat can be easily determined by controlling the amount of the moisture of the solid biomass and the pressure when bringing the solid biomass into contact with the ammonia gas. Accordingly, the saccharification pretreatment of the solid biomass can be controlled simply, easily and stably.

By decompressing the solid biomass and the ammonia gas in contact and recovering the ammonia gas dissolved in the moisture of the solid biomass through vaporization, the ammonia gas is reusable, so that costs for the saccharification pretreatment of the solid biomass are reducible.

In the pressure-resistant ammonia gas reactor 11, the solid biomass is in contact with the ammonia gas at a pressure higher than atmospheric pressure.

With this arrangement, it is only required to supply the ammonia gas stored at the pressure higher than atmospheric pressure in the ammonia gas holder 36 to the ammonia gas reactor 11 into which the solid biomass is fed. Accordingly, a structure for bringing the solid biomass into contact with the ammonia gas at the pressure higher than atmospheric pressure can be easily obtained, thereby facilitating a contact treatment to perform a stable saccharification pretreatment with a simple structure.

The solid biomass having a moisture content in a range of 30 mass % to 95 mass % is used.

Accordingly, the ammonia gas in contact with the solid biomass can be favorably dissolved in the moisture of the solid biomass, which leads to a favorable modification of the solid biomass.

Ammonia gas having a moisture content of 10 mass % or less is used for contact with the solid biomass.

With this arrangement, since an amount of heat required for favorably modifying the solid biomass is obtained by absorption heat generated when the ammonia gas is dissolved in the moisture of the solid biomass, it is unnecessary to heat the solid biomass by a heating unit, or an amount of energy consumption for heating the solid biomass by a heating unit is significantly reducible, so that treatment costs for modifying the solid biomass is reducible and the solid biomass is efficiently modifiable.

The pressure to bring the solid biomass into contact with the ammonia gas is in a range of 0.5 MPa to 4 MPa.

With this arrangement, it is unnecessary to heat the solid biomass to temperatures higher than required, so that disadvantages such as excessive decomposition by heating the solid biomass to higher temperatures are not caused. Additionally, since the solid biomass is sufficiently modified by contacting with ammonia gas under compression, the saccharification treatment can be efficiently performed in the posterior process.

The temperature to bring the solid biomass into contact with the ammonia gas is in a range of 50 degrees C. to 200 degrees C.

Accordingly, the solid biomass can be sufficiently modified without excessive decomposition, so that loss of the solid biomass as a saccharification material is reduced and inhibition to fermentation in the posterior process of the saccharification treatment is reduced.

The solid biomass in contact with the ammonia gas is rapidly decompressed to be blasted.

Accordingly, the solid biomass can be sufficiently modified, so that an efficiency for the saccharification treatment in the posterior process can be improved.

The solid biomass is decompressed to a pressure lower than atmospheric pressure.

With this arrangement, a difference between the pressure applied on the solid biomass in contact with the ammonia gas and the pressure to which the applied pressure is reduced becomes large, so that the effect to blast the solid biomass can be increased to more favorably modify the solid biomass.

Moreover, when the evaporated ammonia gas is cooled and recovered as liquid ammonia, since the non-liquefied ammonia gas is absorbed in water and distilled to be concentrated, and further cooled and recovered as liquid ammonia, the ammonia gas can be recovered at a high efficiency to prevent increase in the treatment costs.

Further, since the modified solid biomass is dried to separate and remove the adsorbed ammonia gas, the ammonia gas can be recovered at a higher efficiency to prevent increase in the treatment costs.

Since the cellulose-based biomass and the starch-based biomass are used as a treatment target, a modification state more favorable than one obtained by a conventional method can be obtained and the efficiency for the saccharification treatment can be improved.

### Modifications

It should be noted that the embodiment described above is only an exemplary embodiment of the invention. The invention is not limited to the above-described embodiment but includes modifications and improvements as long as the object and the advantages of the aspect of the invention can be attained.

Further, the specific arrangements and configurations may be altered in any manner as long as the modifications and improvements are compatible with the invention.

For instance, for supplying ammonia gas to be contacted with the solid biomass, the ammonia gas holder heats and vaporizes liquid ammonia to ammonia gas and supplies the ammonia gas to the ammonia gas reactor at the pressure higher than atmospheric pressure, thereby contacting the ammonia gas with the solid biomass at the pressure higher than atmospheric pressure. However, ammonia gas may be supplied by any arrangement. Specifically, ammonia gas may be supplied by an ammonia gas bottle and the like. Moreover, ammonia gas stored in a gas state may be supplied, or ammonia gas generated by heating an ammonia aqueous solution may be supplied.

Although the recovered ammonia gas is exemplarily recycled, the separated ammonia gas may be filled in an ammonia gas bottle and recovered (i.e., a batch treatment).

Although the solid biomass in contact with the ammonia gas at the pressure higher than atmospheric pressure is exemplarily subjected to a rapid decompression by the decompressor to blast the solid biomass, the solid biomass may be gradually decompressed instead of a rapid decompression. It should be noted that a rapid decompression is preferable in terms of a large blast effect.

Although the pressure applied on the solid biomass is reduced to the pressure lower than atmospheric pressure, the pressure may be reduced to atmospheric pressure. For reducing the pressure to the pressure lower than atmospheric pressure, a vacuum pump is required, which is occasionally disadvantageous in terms of the energy efficiency. It should be noted that, when the applied pressure is defined as one lower than atmospheric pressure, the difference between the applied pressure and the pressure to which the applied pressure is reduced becomes large, so that a large blast effect is easily obtainable.

Specific structures and shapes for implementing the present invention may be other ones as long as the object of the present invention can be attained.

### EXAMPLES

Next, a saccharification pretreatment method for the solid biomass of the invention will be specifically described with reference to Examples. It should be noted that the invention is not limited to the following examples.

### Example 1

Saccharification Pretreatment of Cellulose-Based Biomass

100 g of a bagasse sample (cellulose-based biomass) having a moisture content of 58.7 mass % was put into a 1000-ml volume ammonia gas reactor. Ammonia gas was supplied from an ammonia gas holder to the ammonia gas reactor and was compressed to 2 MPaG. At this time, the temperature was slowly increased. When the pressure reached 2 MPa in two minutes later, the temperature reached 99 degrees C. This condition was kept for 60 minutes. An average temperature during this time was 83 degrees C.

Subsequently, the bagasse sample was supplied to a decompressor of −0.08 MPaG and was rapidly decompressed to be blasted.

After ammonia in a gaseous phase was removed, bagasse was taken out and dried in the shade, whereby ammonia was fully removed. The moisture content of bagasse at this time was 49.6 mass %.

### Saccharification Treatment

0.7 g of bagasse obtained by the saccharification pretreatment was put into a vessel, to which 10 ml of 100-mM sodium acetate buffer prepared at pH 5.0 and 0.2 mol of an enzyme (Accellerase Duet: manufactured by Genencor) were added. The obtained mixture was subjected to saccharification reaction for 72 hours at 120 rpm in a shaker of a constant-temperature bath that was kept at 50 degrees C. Subsequently, an undissolved residue was removed by a centrifuge. The obtained supernatant was measured in terms of a sugar concentration. The sugar concentration was measured by measuring absorbance of 2=490 nm of phenol sulfate according to a colorimetric method using a spectrophotometer (manufactured by Shimadzu Corporation, model: UV-1700).

The sugar concentration was evaluated by comparing monosaccharide ratios each obtained as a mass of the produced monosaccharide per 1 g of a mass of dried bagasse after the saccharification pretreatment. Results are shown in Table 1.

### Example 2

Example 2 was performed in the same manner as in Example 1 except that the holding time in the saccharification pretreatment in Example 1 was changed to 15 minutes. Results are shown in Table 1.

### Example 3

Example 3 was performed in the same manner as in Example 1 except that the pressure in the saccharification pretreatment in Example 1 was changed to 1 MPaG. Results are shown in Table 1.

### Example 4

Example 4 was performed in the same manner as in Example 1 except that bagasse in the saccharification pretreatment in Example 1 was changed to bagasse having a moisture content of 50.2 mass %. Results are shown in Table 1.

### Example 5

Example 5 was performed in the same manner as in Example 1 except that bagasse in the saccharification pretreatment in Example 1 was changed to bagasse having a moisture content of 50.2 mass % and the holding time was changed to 15 minutes. Results are shown in Table 1.

### Example 6

Example 6 was performed in the same manner as in Example 1 except that bagasse in the saccharification pretreatment in Example 1 was changed to bagasse having a moisture content of 50.2 mass % and the pressure was changed to 1 MPaG. Results are shown in Table 1.

### Example 7

Example 7 was performed in the same manner as in Example 1 except that bagasse in the saccharification pretreatment in Example 1 was changed to bagasse having a moisture content of 28.6 mass %. Results are shown in Table 1.

### Example 8

Example 8 was performed in the same manner as in Example 1 except that bagasse in the saccharification pretreatment in Example 1 was changed to bagasse having a moisture content of 28.6 mass % and the holding time was changed to 15 minutes. Results are shown in Table 1.

### Example 9

Example 9 was performed in the same manner as in Example 1 except that bagasse in the saccharification pretreatment in Example 1 was changed to bagasse having a moisture content of 28.6 mass % and the pressure was changed to 1 MPaG. Results are shown in Table 1.

### Example 10

Example 10 was performed in the same manner as in Example 1 except that bagasse in the saccharification pretreatment in Example 1 was changed to bagasse having a moisture content of 2.9 mass %. Results are shown in Table 1.

### Example 11

Example 11 was performed in the same manner as in Example 10 except that the amount of bagasse in the saccharification treatment in Example 10 was changed from 0.7 g to 0.438 g. In other words, the amount of bagasse was prepared to be equal to a weight of dried bagasse subjected to enzyme saccharification in Comparative 1 described later. Results are shown in Table 2.

### Comparative 1

Without the pretreatment of Example 1, the same saccharification treatment using an enzyme as in Example 11 was performed using 0.4 g of dried bagasse. Results are shown in Table 2.

TABLE 1

| | | | | | | monosaccharide | |
|---|---|---|---|---|---|---|---|
| | | | | bagasse moisture | bagasse moisture | concentration in | |
| | Temperature | | | content before | content after | a solution | mono- |
| | Average/ | | | saccharification | saccharification | after enzyme | saccharificatton |
| | Maximum | Pressure | Time | pretreatment | pretreatment | saccharification | ratio |
| Examples | [° C.] | [MPa] | [min] | [%] | [%] | [g/L] | [g/g] |
| 1 | 83/99 | 2 | 60 | 58.7 | 49.6 | 22.4 | 0.64 |
| 2 | 88/101 | 2 | 15 | 58.7 | 38.6 | 23.6 | 0.55 |
| 3 | 75/98 | 1 | 60 | 58.7 | 66.7 | 14.7 | 0.63 |
| 4 | unmeasured | 2 | 60 | 50.2 | 46.0 | 23.3 | 0.62 |
| 5 | 84/103 | 2 | 15 | 50.2 | 39.6 | 17.2 | 0.41 |
| 6 | 81/84 | 1 | 60 | 50.2 | 43.7 | 22.9 | 0.58 |
| 7 | 64/71 | 2 | 60 | 28.6 | 16.9 | 24.1 | 0.41 |
| 8 | 81/101 | 2 | 15 | 28.6 | 17.8 | 18.9 | 0.33 |
| 9 | 74/97 | 1 | 60 | 28.6 | 13.3 | 22.2 | 0.37 |
| 10 | 68/71 | 2 | 60 | 2.9 | 8.7 | 18.6 | 0.29 |

TABLE 2

| | | | monosaccharide |
|---|---|---|---|
| | amount of | amount of dried | concentration in |
| | bagasse used for | bagasse used for | a solution after |
| | saccharification | saccharification | enzyme saccharifi- |
| | treatment [g] | treatment [g] | cation [g/L] |
| Example 11 | 0.438 | 0.40 | 10.8 |
| Comp. 1 | 0.40 | 0.40 | 8.8 |

### Example 12

Saccharification Pretreatment of Starch-Based Biomass

Frozen raw cassava pulp was defrosted with a hot water of 30 degrees C. Moisture of the cassava pulp was blown and dried by a drier at 60 degrees C., and then stored. Subsequently, water was added to the stored dried cassava pulp such that the moisture thereof reached 70 mass %. The obtained cassava pulp was held for several hours in a plastic bag such that the moisture evenly spread and then was subjected to an experiment.

200 g of the above cassava pulp sample having a moisture content of 70 mass % was put into a 1000-ml volume ammonia gas reactor. Ammonia gas was supplied from the ammonia gas holder to the ammonia gas reactor and was compressed to 1 MPaG. At this time, the temperature was slowly increased. When the pressure reached 1 MPaG in two minutes later, the temperature reached 95 degrees C. This condition was kept for 15 minutes. An average temperature during this time was 80 degrees C.

Subsequently, the cassava pulp sample was supplied to a decompressor of −0.08 MPaG and was rapidly decompressed to be blasted. After ammonia in a gaseous phase was removed, the cassava pulp was taken out and dried in the shade, whereby ammonia was fully removed. A moisture content of the cassava pulp at this time was 49.6 mass %.

When the starch amount in the cassava pulp before the saccharification treatment was measured using a total starch measurement kit (manufactured by Megazyme), a result was 63.5 mass % (based on dried cassava pulp).

Saccharification Treatment

1 g of the cassava pulp obtained by the above saccharification pretreatment was put into a vessel, to which 10 ml of 100-mM sodium acetate buffer prepared at pH 5.0, 0.1 mol of an amylase enzyme (Bacillus amyloliquefaciens: manufactured by Sigma-Aldrich Co.) and 0.05 ml of a glucoamylase enzyme (Aspergillus niger: manufactured by Sigma-Aldrich Co.) The obtained mixture was subjected to a saccharification reaction for 72 hours at 120 rpm in a shaker of a constant-temperature bath that was kept at 60 degrees C.

Subsequently, an undissolved residue was removed by a centrifuge. The obtained supernatant was measured in terms of a sugar concentration. The sugar concentration was measured by measuring absorbance of λ=520 nm of a free reducing sugar according to a colorimetric method using Somogyi-Nelson method with a spectrophotometer (manufactured by Shimadzu Corporation, model: UV-1700).

As a result, a starch saccharification efficiency obtained from the monosaccharide concentration in the solution after the enzyme saccharification was 76.8%. When a value obtained by converting a total starch concentration of the cassava pulp to a glucose concentration was defined as 100%, the starch saccharification efficiency was calculated based on a ratio of the value of the reducing sugar produced in the above saccharification treatment. Herein, the free sugar contained in the cassava pulp was subtracted from the value of the produced reducing sugar in advance.

### Results

The results in Table 1 show that the monosaccharide ratios were increased by performing the saccharification pretreatment of the invention.

It is also recognized in Examples that the monosaccharide ratio tends to be increased in accordance with a higher moisture content of bagasse (material before the saccharification pretreatment). This is considered because blast effects were increased by the increased amount of dissolving ammonia.

Under the same moisture concentration of the material, it is recognized that the monosaccharide ratio is improved in accordance with a higher pressure and a longer treatment duration of time. Specifically, the highest monosaccharide ratios are respectively obtained in Example 1 among Examples 1 to 3, Example 4 among Examples 4 to 6, and Example 7 among Example 7 to 9.

On the other hand, from the results shown in Table 2, the sugar concentration in Example 11 in which the saccharification pretreatment of the invention was performed is higher than the sugar concentration in Comparative 1 in which the saccharification pretreatment is not performed. Since the dried mass is the same as those in Example 11 and Comparative 1, it is recognized that activity of the enzyme saccharification is improved by performing the saccharification pretreatment of the invention.

### INDUSTRIAL APPLICABILITY

The invention is applicable to a pretreatment method for modifying solid biomass containing cellulose or starch for a saccharification treatment.

### EXPLANATION OF CODES

1: saccharification pretreatment apparatus
10: ammonia gas contact section
11: ammonia gas reactor
20: decompression section
30: ammonia gas recovery section
40: ammonia gas removing section

1. A saccharification pretreatment method of solid biomass, comprising:
contacting moisture-containing solid biomass with ammonia gas at a pressure higher than atmospheric pressure to dissolve the ammonia gas into moisture of the solid biomass;
decompressing the solid biomass contacted with the ammonia gas to a pressure lower than a pressure in the contacting with the ammonia gas to evaporate the ammonia gas dissolved in the moisture of the solid biomass; and
separating to recover the ammonia gas evaporated in the decompressing from the solid biomass.

2. The saccharification pretreatment method of solid biomass according to claim 1, wherein
in the contacting of the solid biomass with the ammonia gas,
the solid biomass is fed into a pressure vessel,
the ammonia gas is compressed and supplied into the pressure vessel in which the solid biomass is fed, and
the solid biomass and the ammonia gas are contacted with each other at a pressure higher than atmospheric pressure.

3. The saccharification pretreatment method of solid biomass according to claim 1, wherein
the solid biomass has a moisture content in a range of 30 mass % to 95 mass %.

4. The saccharification pretreatment method of solid biomass according to claim 1, wherein
the ammonia gas has a moisture content of 10 mass % or less.

5. The saccharification pretreatment method of solid biomass according to claim 1, wherein
in the contacting of the solid biomass with the ammonia gas, the solid biomass is contacted with the ammonia gas at a pressure in a range of 0.5 MPa to 4 MPa.

6. The saccharification pretreatment method of solid biomass according to claim 1, wherein
in the contacting of the solid biomass with the ammonia gas, before performing the decompressing, a temperature of the solid biomass is set in a range of 50 degrees C. to 200 degrees C.

7. The saccharification pretreatment method of solid biomass according to claim 1, wherein
in the decompressing, the solid biomass is rapidly decompressed to be blasted.

8. The saccharification pretreatment method of solid biomass according to claim 1, wherein
in the decompressing, the pressure of the solid biomass is decompressed to a pressure lower than atmospheric pressure.

9. The saccharification pretreatment method of solid biomass according to claim 1, further comprising:
recovering the ammonia gas by heating to dry the solid biomass from which the ammonia gas is separated in the separating and separating ammonia adsorbed to the solid biomass.

10. The saccharification pretreatment method of solid biomass according to claim 1, wherein
the solid biomass is a cellulose-containing biomass.

11. The saccharification pretreatment method of solid biomass according to claim 1, wherein
the solid biomass is a starch-containing biomass.

12. A saccharification pretreatment apparatus of solid biomass, comprising:
an ammonia gas contact section that contacts a moisture-containing solid biomass with ammonia gas at a pressure higher than atmospheric pressure to dissolve the ammonia gas into moisture of the solid biomass;
a decompressor that decompresses the solid biomass contacted with the ammonia gas in the ammonia gas contact section to a pressure lower than a pressure in the ammonia gas contact section to evaporate the ammonia gas dissolved in the moisture of the solid biomass; and
an ammonia gas separating section that separates to recover the ammonia gas evaporated in the decompressor from the solid biomass.

13. A saccharification method of the solid biomass treated with the saccharification pretreatment method of solid biomass according to claim 1.
